(19) United States
(12) Patent Application Publication
(10) Pub. No.: US 2009/0317475 A1
(43) Pub. Date: Dec. 24, 2009

(54) COMBINATION ANTITUMOR THERAPIES

(76) Inventors: Robert A. Beardsley, St. Louis, MO (US); Nickols G. Allen, Wentzville, MO (US)

Correspondence Address: MORRISON & FOERSTER LLP, 12531 HIGH BLUFF DRIVE, SUITE 100, SAN DIEGO, CA, 92130-2040, US

(21) Appl. No.: 12/160,091

(22) PCT Filed: Jan. 3, 2007
(86) PCT No.: PCT/US07/00083

§ 371 Date: Nov. 3, 2008

Related U.S. Application Data
(60) Provisional application No. 60/756,048, filed on Jan. 3, 2006.

(51) Int. Cl. A61K 9/14; A61P 35/00; A61K 31/336; A61K 31/337; A61K 39/395

(52) U.S. Cl. 424/489; 514/475; 514/449; 424/158.1; 424/133.1; 977/773; 977/906

(57) ABSTRACT

Improved methods of treating cancers that are characterized by unwanted angiogenesis employ combinations of therapies designed to inhibit the VEGF mediated angiogenesis pathway with therapies designed to inhibit the bFGF-mediated angiogenesis pathway.

### RELATED APPLICATION

This application claims benefit of provisional application U.S. Ser. No. 60/756,048 filed 3 Jan. 2006 which is incorporated herein by reference in its entirety.

### TECHNICAL FIELD

The invention relates to combination therapies targeting more than one angiogenic pathway for the treatment of cancer. Specifically, the invention concerns combining anti-vascular endothelial growth factor (VEGF) pathway therapies with therapies directed against basic fibroblast growth factor (bFGF) mediated angiogenesis.

### BACKGROUND ART

Anti-VEGF therapies such as Avastin® (an anti-VEGF Mab), VEGF receptor antagonists or signaling inhibitors (tyrosine kinase inhibitors) all cause “normalization” of the angiogenic vasculature in tumor-bearing subjects. The net results include more normal-appearing vasculature upon histological exam, decreased interstitial fluid pressure (IFP), enhanced oxygenation of the tumor core with increased blood flow, enhanced responsiveness to radiation due to increased oxygen delivery, augmented blood flow and delivery of chemotherapeutic agents to tumor core. Thus, additive or synergistic effects of anti-VEGF pathway therapy with radiation or chemotherapy on reducing tumor volume, leading to tumor regression or stable disease have been observed.

Therapies directed against the bFGF mediated angiogenesis pathway may target the integrin α_{ν}β₃, which characterizes activated cells involved in angiogenesis. Inhibition of α_{ν}β₃-supported angiogenesis and potentially vascular normalization are believed to occur by reduction of an angiogenesis pathway mediated by bFGF, that is distinct from the VEGF pathway. Blockage of α_{ν}β₃ per se may be inadequate either because of functional redundancy in the role of α_{ν}β₃ in the bFGF pathway, so that pathway may remain active even with complete blockade of this integrin, or because the bFGF pathway itself is not an exclusive mediator of angiogenesis.

Another approach to therapy believed to target the bFGF-mediated pathway employs α_{ν}β₃-targeted delivery of an antiproliferative agent. One embodiment of this approach is described in PCT publication WO 03/062198 to Lanza, et al. In this embodiment, peptidomimetic compounds that bind βᵥβ₃ integrin are coupled to perfluorocarbon-based nanoparticles which further comprise an antiproliferative agent.

### DISCLOSURE OF THE INVENTION

Because the VEGF and bFGF angiogenesis pathways represent two complementary, but at least partially independent pro-angiogenic pathways, combinations of agents resulting in suppression of, or selection against, both pathways offers more effective anti-angiogenic activity than inhibiting either alone. Such a combination regimen either by itself or along with “standard of care” (SOC) chemotherapy for appropriate tumor types (i.e., tamoxifen for breast cancer) would provide effective treatment. Such a combination regimen with radiation therapy also increases efficacy of radiotherapy. Because angiogenesis has also been implicated in the rapid growth of “satellite” tumors, as well as metastatic spread and recurrence of cancer, upon surgical resection of primary tumors, such a combination regimen would also serve as an adjuvant to surgical resection.

Thus, in one aspect, the invention is directed to a method to treat malignancies characterized by angiogenesis in a subject, which method comprises administering to a subject in need of such treatment a cancer-inhibiting amount of a therapy directed to the inhibition of the vascular endothelial growth factor (VEGF) angiogenesis pathway as well as a cancer-inhibiting amount of a therapy directed against the basic fibroblast growth factor (bFGF) mediated pathway of angiogenesis. Therapies directed against the bFGF mediated pathway, in particular, may employ an α_{ν}β₃-targeted antiproliferative agent.

In further aspects, the invention is directed to methods to treat tumors which combine the foregoing with either radiation therapy or chemotherapy or both, and to kits containing medicaments for use in the method.

### MODES OF CARRYING OUT THE INVENTION

A wide range of inhibitors of the VEGF pathway is known in the art. The monoclonal antibody Avastin® marketed by Genentech is approved for clinical use. Other antibodies directed against VEGF are also available, as well as are compounds that antagonize the VEGF receptor. An alternative group of therapies that inhibit VEGF pathway relies on inhibiting the signaling pathway associated with the VEGF receptor, notably compounds that inhibit tyrosine kinases. Another alternative comprises use of compounds that antagonize tumor-associated cells expressing VEGF receptor or the relevant integrin. The methods of the invention employ one or more of such anti-VEGF pathway therapies.

Antibodies useful as inhibitors may target either VEGF or the VEGF receptor, or an appropriate integrin. “Antibodies” as defined in the present application include both polyclonal and monoclonal antibodies which may be chimeric—i.e., contain constant regions from one species and variable regions from another, or may be humanized—i.e., altered in the variable region to more closely mimic human antibodies and comprising human constant regions. “Antibodies” also include fragments which remain immunoreactive with VEGF or its receptor, such as F_{ab}, F_{(ab′)2} fragments or maybe recombinantly produced single chain forms such as Fᵥ or Fₛᵥ forms. Any portion or form of an antibody that retains the relevant immunoreactivity is included within the definition of “antibodies.” Means for producing antibodies as defined above are well known in the art.

In addition to antibodies, small molecules that antagonize the VEGF receptor or that inhibit the signaling pathway associated therewith are well known in the art. Such inhibitors include, for example, SU5416, GW654652 and ZD6474. Small molecules that inhibit this receptor continue to be developed.

In addition to these direct approaches, targeted antiproliferative agents or other toxic agents that are directed to cells involved in the VEGF-mediated pathway may be employed. Conjugates of toxins or antiproliferative compounds to agents targeting the VEGF receptor may be employed. In addition, cells associated with the VEGF-mediated pathway display the βᵥβ₅ integrin, and targeting the antiproliferative or toxic compound to this integrin is also effective. As is the case with regard to targeting the bFGF-mediated pathway described below, a preferred method of such targeting employs delivery vehicles that contain the antiproliferative agent or toxin and which comprise a targeting moiety to either the receptor or the integrin.

Similarly, inhibitors of the bFGF pathway are well known. These, too, may be antibodies directed against either bFGF or its receptor or its integrin or may be antagonists of the bFGF receptor or of other receptors in the pathway, and the like. “Antibodies” are as defined above with respect to the VEGF pathway—i.e., any antibody or portion thereof that retains its immunoreactivity with bFGF or the bFGF or relevant integrin receptor is included. Small molecule antagonists of the bFGF receptor are also known, including a synthetic compound of the pyrido[2,3-d]pyrimidine class, designated PD173074. This inhibitor inhibits the tyrosine kinase activities of the VEGF receptor as well.

The integrin associated with the bFGF angiogenesis pathway is α_{νβ}₃, and in a manner analogous to that set forth for the VEGF pathway above, toxins or antiproliferative agents may be targeted to this pathway either by coupling them to a moiety that binds the bFGF receptor or by targeting them to the α_{νβ}₃ integrin. As described above, one approach to such targeting is to couple a targeting agent to delivery vehicles that contain the toxin or antiproliferative agent. Compositions that deliver antiproliferative or toxic agents selectively to activated cells characterized by the α_{ν}β₃ integrin are described in detail in PCT publication WO 03/062198 cited above.

Suitable antiproliferative agents include, for example, taxanes such as paclitaxel and docetaxel; fumagillin; anthracyclines such as daunorubicin, doxorubicin and epirubicin; camptothecins such as irinotecan and topotecan; vincristine, and a wide variety of other such drugs, many known as chemotherapeutic agents, such as carboplatin; cisplatin; cyclophosphamide; mitomycin C; mitoxantrone; 5-fluorouracil (5-FU); FUDR; gemcitabine; methotrexate; bleomycin; etoposide; vinblastine; vindesine; vinorelbine; and genistein.

One embodiment of the compositions designed to inhibit the VEGF and/or bFGF mediated pathways employs targeted delivery vehicles containing an antiproliferative or toxic agent that are targeted to the relevant receptor or to the relevant integrin. Such delivery vehicles may include liposomes, micelles, polymer-based particles, or nanoparticles of various structures. One important structure useful in the invention is that of nanoparticles that contain fluorocarbon cores, in particular fluorocarbon cores that remain liquid at body temperature and during delivery of the associated drug, which fluorocarbon cores are coated with a lipid/surfactant coating that permits coupling of the targeting agent to the particles and effective delivery of drugs carried by the particles. Such particles are described in detail in U.S. Pat. No. 6,821,506; U.S. Pat. No. 6,548,046; and European patent 831933, as well as the above cited PCT publication. Briefly, a relatively high boiling perfluorocarbon such as perfluorooctylbromide is emulsified in the presence of surfactant/lipid such as a composition of phosphatidyl ethanolamines to obtain nanoparticulate compositions. Means for coupling targeting agents to the particles by employing compounds that imbed in the lipid/surfactant layer are described in these documents and in PCT publication WO 2004/067483 to Lanza, et al. Various such methods are now known in the art.

Thus, in one embodiment, compositions useful in the invention comprise nanoparticulate delivery vehicles that are directly coupled to an agent that targets the desired receptor or integrin. Such targeting agents include antibodies as described above, as well as peptides and peptidomimetics.

One embodiment comprises use of Avastin® as the anti-VEGF pathway therapy. One embodiment employs a particulate carrier targeted to α_{ν}β₃ as the anti-bFGF pathway therapy. One peptidomimetic targeting βᵥβ₃ comprises the moiety:

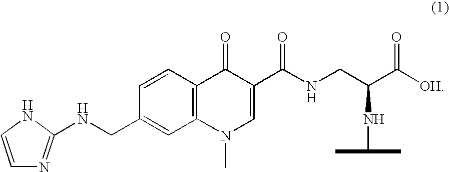

This moiety is coupled to a diacyl phosphatidyl ethanolamine, e.g., through a linker. The moiety of formula (1) may be coupled, e.g., to 1,2-Distearoyl-sn-Glycero-3-Phosphoethanolamine-N-[Maleimide(Polyethylene Glycol)2000] through an additional linker to obtain, for example,

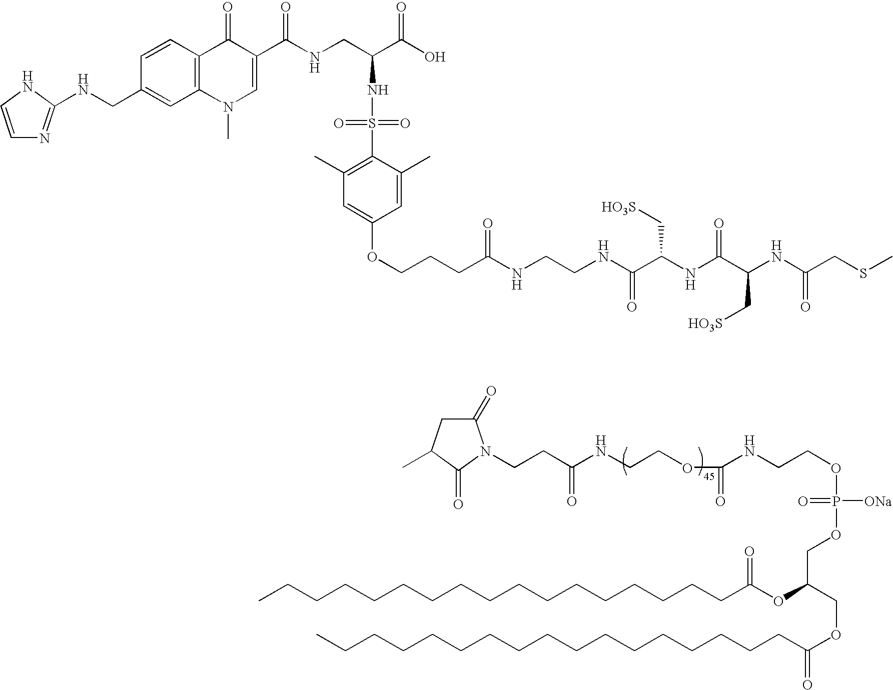

or to obtain

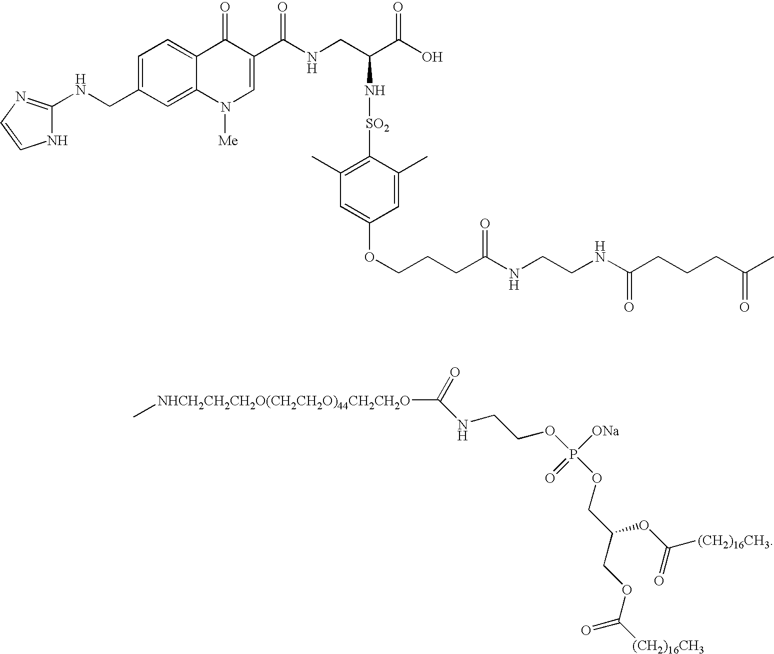

Modified forms of the linking moieties may also be employed containing fewer or more methylene units. Thus, the targeting moiety associated with the nanoparticles has the formula

targeting agent-SO₂-phenylene-O(CH₂)ₙCONH(CH₂)ₙNHCO(CH₂)ₙCONH(CH₂)ₙO(CH₂CH₂)ₓCH₂CH₂OCZ-anchor

wherein each n is independently 1-4;

x is an integer of 20-60;

Z is NH or O;

the targeting agent is a moiety containing an amino group or a hydroxyl group for coupling to the remainder of the molecule; and

the anchor is a lipophilic compound for embedding the compound in a lipid/surfactant base composition. The “targeting agent” is preferably of formula (1).

The foregoing embodiments may be combined in the practice of the invention.

A “cancer-inhibiting amount” of an individual component in the combination means an amount that, together with the other component in the combination, results in cancer inhibition. In one embodiment the inhibition of tumor growth is additive or synergistic for the combination.

The combination therapies of the invention may be administered by appropriate routes, depending on the nature of the therapy chosen. Typically, administration is systemic, by injection, for example, by intravenous injection. However, other systemic methods of administration may also be employed, including oral, transdermal or transmucosal administration, and other forms of injection. Parenteral routes are preferred. Local administration may also be employed using, for example, catheters or implants. The formulation selected will depend on the mode of administration and suitable formulations for pharmaceuticals including antibodies, proteins and small molecules are readily available in the art. A formulary for pharmaceuticals covering a variety of approaches is found in Remington's Pharmaceutical Sciences, latest edition, Mack Publishing Co., Easton, Pa., incorporated herein by reference.

In the therapies of the invention, the VEGF pathway based inhibition therapy and the other component of the combination, the anti-bFGF pathway based therapy, may be administered in tandem, simultaneously, or in any spaced regimen protocol. Either therapy of the combination may be administered prior to the other. If more than one therapy directed against the VEGF and/or bFGF mediated pathways is included, the pattern of individual therapeutic methods can be varied according to the judgment of the attending practitioner. The modes of administration may be identical, similar or different.

The dosage levels of each are such that an effective antitumor result is obtained from the combination. Dosage levels and protocols may be optimized and are dependent on the judgment of the practitioner as well as the parameters characterizing the individual subject and the subject's condition.

The combination therapies of the invention may further be combined with radiation therapy, chemotherapy or both. The sequence of administering such treatments may be sequential or substantially simultaneous and in any order. The combination therapies of the invention may also be used as an adjuvant to surgical resection of a tumor, to reduce or prevent growth of satellite disease, metastatic spread or recurrence of the cancer.

The components for use in the combination therapies may be packaged in convenient kit form, with appropriate dosage levels premeasured if desired. For example, measured quantities of one or both types of therapeutic components may be packaged in syringes for immediate administration.

The following examples are offered to illustrate but not to limit the invention.

### EXAMPLE 1

A murine tumor model wherein the tumor is responsive to Avastin® and to paclitaxel is employed. The α_{ν}β₃-targeting moiety is embedded in the lipid/surfactant using a compound of the formula

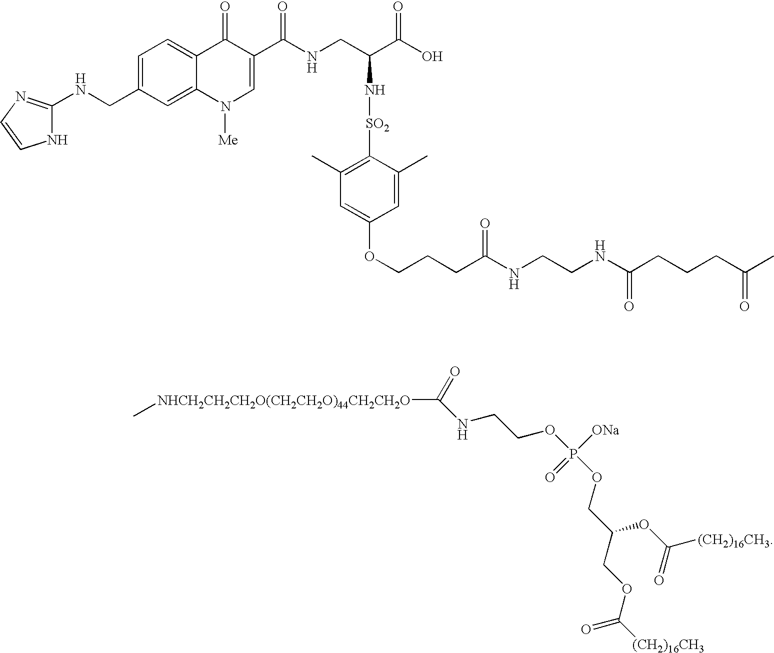

Emulsions of paramagnetic perfluorocarbon nanoparticles targeted to α_{ν}β₃-integrins are prepared as described in Winter, et al., Circulation (2003) 108:2270-2274. In general, the nanoparticulate emulsions are comprised of 20% (v/v) perfluorooctylbromide (PFOB; Minnesota Manufacturing and Mining), 2% (w/v) safflower oil, 2% (w/v) of a surfactant co-mixture, 1.7% (w/v) glycerin and water for balance. The surfactant co-mixture includes 58 mole % lecithin (Avanti Polar Lipids, Inc.), 10 mole % cholesterol (Sigma Chemical Co., St. Louis, Mo.), 0.1 mole % peptidomimetic of formula (1) conjugated to PEG2000-phosphatidylethanolamine (Avanti Polar Lipids, Inc.) as shown above and 31.8 mole % phosphatidylethanolamine (Avanti Polar Lipids, Inc.).

Nanoparticulate formulations containing paclitaxel included 0.2 mole % of paclitaxel in the surfactant mixture at the proportionate expense of lecithin.

The surfactant components are prepared as described in Lanza, et al., Circulation (2002) 106:2842-2847 and in Winter, et al., Circulation (2003) 108:2270-2274, combined with PFOB, safflower oil and distilled deionized water. The mixture is emulsified in a M110S Microfluidics emulsifier (Microfluidics, Inc, Newton, Mass.) at 20,000 PSI for four minutes. Particle sizes are determined at 37° C. with a laser light scattering submicron particle analyzer (Malvern Instruments, Malvern, Worcestershire, UK).

The tumor afflicted animals are treated with multiple doses of Avastin® and the above-described composition comprising α_{ν}β₃-targeted liquid perfluorocarbon based nanoparticles coated with lipid/surfactant comprising paclitaxel.

The effect of the combination on tumor progression is monitored.

1. A method to treat malignancies characterized by angiogenesis in a subject, which method comprises administering to a subject in need of such treatment
(a) a cancer-inhibiting amount of a therapy that inhibits the vascular endothelial growth factor (VEGF) angiogenesis pathway (anti-VEGF pathway therapy), and
(b) a cancer-inhibiting amount of a therapy directed against the basic fibroblast growth factor (bFGF) mediated angiogenesis pathway (anti-bFGF pathway therapy).

2. The method of claim 1, wherein the anti-bFGF pathway therapy comprises administering an α_{ν}β₃-targeted antiproliferative agent.

3. The method of claim 2, wherein the α_{ν}β₃-targeted antiproliferative agent comprises nanoparticulate delivery vehicles coupled to ligand specific for α_{ν}β₃ integrin, which delivery vehicles further comprise said antiproliferative agent.

4. The method of claim 3, wherein the delivery vehicles comprise liquid perfluorocarbon cores coated with lipid/surfactant.

5. The method of claim 1, wherein the antiproliferative agent is a taxane or fumagillin.

6. The method of claim 1, wherein the anti-VEGF pathway therapy comprises administering antibodies immunoreactive with VEGF, VEGF receptor antagonists and/or VEGF signaling inhibitors.

7. The method of claim 6, wherein the anti-VEGF pathway therapy comprises administering Avastin®.

8. The method of claim 1, which further comprises administering chemotherapy and/or radiation therapy.

9. The method of claim 1, wherein the treatment is administered after surgical resection of a malignant tumor to prevent or reduce growth of other tumors, metastatic spread of the malignancy, or recurrence of the malignancy.
